(19) United States
(12) Patent Application Publication
(10) Pub. No.: US 2008/0227144 A1
(43) Pub. Date: Sep. 18, 2008

(54) Tissue sample support and orientation device

(76) Inventor: Stephen D. Nightingale, Bethesda, MD (US)

Correspondence Address: DUANE MORRIS LLP, Suite 1000, 505 9th Street, N.W., Washington, DC, 20004, US

(21) Appl. No.: 12/073,599

(22) Filed: Mar. 7, 2008

Related U.S. Application Data
(60) Provisional application No. 60/905,906, filed on Mar. 9, 2007.
(60) Provisional application No. 60/906,506, filed on Mar. 13, 2007.

(30) Foreign Application Priority Data
Mar. 7, 2008 (US) PCT/US2008/003028

(51) Int. Cl. C12M 1/26; G01N 33/50

(52) U.S. Cl. 435/40.52; 435/309.1

(57) ABSTRACT

Disclosed is a tissue sample support and orientation device comprising: (i) a base member formed of one or more materials which can be sectioned in a microtome and which is resistant to histological stains and to degradation from chemicals used to fix and process tissue; and (ii) a plurality of support members arranged on the base member in a predetermined spaced relationship, each of the support members having a stem portion and a head portion and formed of one or more materials which can be sectioned in a microtome and which is resistant to histological stains and to degradation from chemicals used to fix and process tissue, wherein the stem portion has a proximal end attached to the base member and a distal end attached to the head portion, and further wherein the head portion is dimensioned to engage and retain a tissue sample during processing thereof.

This application claims the benefit of the prior filing date of U.S. Provisional Patent Application No. 60/905,906, filed Match 9, 2007, and U.S. Provisional Patent Application No. 60/906,506, filed Mar. 13, 2007. The disclosure of each of these prior applications is hereby expressly incorporated by reference in its entirety.

### FIELD OF THE INVENTION

The present invention is in the field of tissue sample analyses, and to the particular field of obtaining, handling and processing tissue biopsy samples.

### BACKGROUND OF THE INVENTION

When disease is suspected in a living being, the physician must arrive at a specific diagnosis. Some disease processes, particularly tumors, require a histologic and/or cytologic diagnosis. While radiologic tools are useful in detecting the presence of a tumor, the cell type of the tumor can only be determined by a pathologist's examination of a histologic or cytologic sample of the tumor.

There are a number of devices that have been fashioned to actually perform the act of taking tissue samples. These devices may obtain tissue for histology or, in the case of needle aspiration biopsies, samples for cytology and histology. In many cases, these samples are very small and difficult to retrieve and process. These small tissue fragments may originate from a punch, or similar biopsy procedure devices or from Fine Needle Aspiration Biopsy (FNAB) biopsies.

FNAB is typical and produces single cells, small cell clumps and fragments which are immediately smeared onto a glass slide (direct smears) or rinsed into a container with preservative fluid. After being transported to the laboratory, these samples are centrifuged onto a glass slide (cytospin smears). In some cases needle aspiration biopsy produces tissue fragments which are large enough to process histologically. If successfully retrieved, these fragments are submitted in blood clot or agar in a technique known as cell block preparation which are then immobilized in wax for sectioning and slide preparation.

FNAB is one example of the tissue collection techniques used and the problems which are of interest to the present invention. Fine Needle Aspiration Biopsy techniques have been practiced for many years and the literature contains many studies on technique and comparison of various improved devices for same.

There exists two different kinds of biopsy needles. Those with active or movable cutting elements and those that are passive or non-moving. Active needles have two basic problems, which are cost and complexity. The needles that are of greatest particular interest to this invention may be as small 22 gauge which is 0.028″ OD and 0.016″ ID. Some prior art designs use an active element down the ID bore to sever and capture tissue. However, 0.016″ does not provide a great deal of clearance for these elements and thus these prior art needles are inefficient and may fragment or otherwise damage the tissue being collected.

Other methods of obtaining samples are also discussed in the literature and also have problems. Each is characterized by tissue size and number of pieces generally available, as well as whether orientation in the eventual sectioning plane is critical. Examples include: (i) Fine Needle Aspiration Biopsy—very small pieces of tissue taken from the core of a fine needle and generally transported in fixative solution; (ii) GI biopsy—characterized by a few small tissue pieces and a need to concentrate the tissue pieces in close proximity to each other; (iii) prostate chips—orientation is irrelevant for these samples; (iv) endometrial curettings—characterized by varying size samples; orientation is irrelevant; (v) vessel—orientation is critical (sections need to be transverse); (vi) core biopsy—orientation is critical (the tissue should lie flat all in the same plane); (vii) gall bladder—orientation is critical (the tissue should be embedded on edge); (viii) uterine wall, breast or large tumors—orientation is not critical (sample lies flat in a plane).

Some of these methods are characterized by the possibility of supplying extremely small tissue samples. Some samples can be as small as a few cells, and extremely small samples can create problems. These problems include loss of the sample, dehydration of the sample, and contamination of the sample during harvesting, storage and transport. Still further, as will be more evident from the following discussion, small samples are extremely difficult and time consuming to process in the laboratory.

Still further, in many cases, a tissue sample is mixed with effluent. Prior art devices and methods account for collection of effluent only and do not provide devices and methods for trapping tissue specimens. The prior art collects effluent, but does not provide devices or methods for the separation of tissue from the effluent. Therefore, there is a need for apparatus and a method for handling effluent as well as tissue samples and for efficiently separating tissue from effluent.

Once a tissue sample is harvested it must be transported to the pathology lab for processing. Currently, handling and processing of small biopsies in the histology laboratory is a tedious task and requires multiple manual manipulations of the specimen. Fine Needle Aspiration Biopsy (FNAB) is typical. Therefore, there is a need to handle and process very small samples of tissue in an expeditious manner.

In addition to the above problems, a further problem with currently used apparatus and methods is associated with the orientation of samples. Currently, in a pathology lab, the pathologist will gross-in the tissue samples, cut them into appropriate size specimens, if necessary, and place them into a tissue cassette for processing. Herein lies one of the biggest problems of the existing art. When the tissue sample is placed into the tissue cassette, the pathologist orients the sample so that any surface in which he or she desires to see sectioned is placed face up in the cassette. The technician who retrieves the tissue from the cassette after processing knows through training that, when opening the cassette, the tissue surface that faces up when first opened is then placed face down into the wax mold, which in turn will become the first surface to be sectioned by a microtome blade. This is an established protocol which is observed in most pathology labs today. This process then necessitates human involvement and redundant handling. In addition, sometimes special sponge materials must be packed into the cassette to keep a sample oriented or to prevent loss from the cassette if it is too small and may turn or lose its orientation during the tissue processing. Sometimes, notes and drawings accompany tissue samples to show how they should be oriented in the wax.

No current system or method provides the ability to maintain critical tissue orientation throughout these steps and eliminate human errors in the associated manual steps and procedures. Therefore, there is a need for a system and a process that can maintain the preferred orientation of the tissue sample from the time of initial gross-in throughout the tissue processing procedure and continuing through the wax embedding stage with no human involvement required beyond initial gross in.

Yet another problem associated with harvesting and handling of tissue samples for biopsy analysis is associated with the analysis process itself. In the analysis procedure, the sample is exposed to heat and chemicals which can cause the tissue and/or its support to change shape and/or move. The sample-holding structure should account for this or there may be a risk of damaging the sample or the sample holder. Accordingly, there is a need for an apparatus for holding a harvested biopsy sample in a manner that accommodates the tissue analysis process.

Another problem encountered with presently available systems is the lack of integration and multiple handling steps required to produce a sectioned sample for pathological examination. Therefore, there is a need for an approach which reduces the time and handling of biopsy samples.

By way of background, a review of the standard procedure that each sample must undergo to get from harvest to a prepared histologic slide is necessary. First, the sample must be taken with the appropriate instrument. The tissue is then retrieved from the instrument and deposited into some sort of specimen container, usually with a fixative such as 10% formalin. The container is labeled and transported to the pathology lab. Herein lies the first problem with the prior art. With no way to control where the sample lodges in the container, the sample may stick to the lid or sides of the container and become dried out before it reaches the pathology lab; rendering it difficult, if not impossible to interpret. In addition, the samples may be extremely small and may be hard to locate and retrieve from the container.

When the pathology laboratory receives the container, the specimen is logged into the manual or computerized anatomic pathology system and is assigned a unique surgical pathology accession number. This number is placed on the specimen container and is subsequently used to label histology slides, cassettes and the final surgical pathology report. The specimen is logged into the paperwork system and physically described in an appropriate medium, such as dictation or the like, by a pathologist or assistant. This is the description portion of the process known as “grossing-in” the specimen.

The grossing-in continues when the pathologist or assistant manually retrieves the specimen and views the specimen, and then sections the specimen into appropriate size morsels, if necessary, and places them into a plastic tissue cassette. If very tiny or multiple, the pieces of tissue must be immobilized within some device such as two layers of sponge or a tea bag to prevent them from escaping from the cassette during processing. Many times a surgeon will have taken diffuse biopsy samples or scrapings from the mucosal lining of an organ, such as an endocervical biopsy. Often these samples are very small and multiple such as is the case with tissue fragments from FNAB. Other times a doctor will deposit the sample in filter paper which resembles a tea bag. All of these various tissue specimens end up in a tissue cassette. As used herein, the term “grossing-in” includes both the description of the tissue sample and the preparation of the tissue sample for further processing.

At the end of the day, all of the cassettes are put into a tissue processor where the tissue is subjected to a sequence of solutions and heat. These solutions gradually replace water in the cells with alcohol, followed by xylene, and ultimately by wax. This gives the wax-impregnated tissue a similar consistency to the wax surrounding the tissue in the next step. After the tissue processing is complete, usually the following morning, the sample is again handled to remove it from the cassette where it is placed and oriented in a mold. At this point if a tea bag or sponge was used to immobilize the sample, the pathology lab is then faced with trying to extract or scrape the wax-impregnated specimen from the paper, before placing the specimen in the wax mold.

An embedding medium such as hot (molten) paraffin wax is poured into the mold to immobilize the tissue in a solid block of wax. Wax or paraffin can be used as an embedding medium; however, agar or even chemically setting resins such as polyester can be used. Harder resins can also be sectioned with a saw blade and then ground and polished to a thin film. After cooling, the wax block is removed from the mold, placed into a microtome and sectioned into thin slices approximately 4-6 microns thick. These sections are floated onto glass slides, stained, cover-slipped, and are then ready for microscopic examination. In this process, samples are handled or transferred many times. Each step in the handling process takes time and human involvement.

Some long thin tissue samples are difficult to align and orient. The prior art includes devices with walls and pegs between which tissue is placed. While in many instances those configurations may work adequately, such as for fallopian tubes, in other instances, such as for gallbladder, it is difficult to place the tissue between the posts.

Once the tissue is properly supported by the orientation device, the device and the tissue are both subjected to the analysis process. Consequently, in addition to being easy to use in connection with biopsy samples, the orientation device must be able to withstand the analysis process and be sectionable as well.

Therefore, there is a need for apparatus and method to improve the harvesting of tissue samples.

### SUMMARY OF THE INVENTION

Accordingly it is an object of the present invention to provide a device that supports and orients a tissue sample for processing and analysis.

It is another object of the present invention to provide a tissue sample support and orientation device that can maintain the preferred orientation of the tissue sample from the time of initial gross-in throughout the tissue processing procedure and continuing through the wax embedding stage with no human involvement required beyond initial gross-in.

It is another object of the present invention to provide a tissue sample support and orientation device for efficiently harvesting tissue samples for biopsy.

It is another object of the present invention to provide a method for handling harvested tissue samples in an efficient manner with a minimum of human intervention.

It is another object of the present invention to provide a tissue sample support and orientation device that can retain tissue samples and facilitate easy transfer of the specimen without having to individually retrieve small tissue fragments from a sample container.

It is another object of the present invention to provide a tissue sample support and orientation device that can be inserted into a tissue cassette, mesh bag, lens paper, sponge or other material(s) during the processing of the immobilized tissue.

It is another object of the present invention to provide a tissue sample support and orientation device that is sectionable and that is constructed of a material that is able to be sectioned in a microtome and appears non-distracting in the histologic sections and does not stain with tissue stains applied to the sections and that is impervious to the harsh chemical and temperature environment involved in processing tissue samples.

It is another object of the present invention to provide a tissue sample support and orientation device which assures that the tissue will be oriented in the desired sectioning plane.

It is another object of the present invention to provide a fine needle aspiration biopsy device which includes a tissue sample support and orientation device specifically adapted for the needs of specimen processing in pathology.

It is another object of the present invention to provide a tissue sample support and orientation device that permits a tissue sample held therein to be sectioned in microtome.

It is another object of the present invention to provide a tissue sample support and orientation device that retains tissue samples in a predetermined orientation during processing.

Still other objects of the present invention are directed to methods of using the foregoing tissue sample support and orientation devices.

In accordance with this and other objects, a first embodiment of the present invention is directed a tissue sample support and orientation device comprising: (i) a base member formed of a material which can be sectioned in a microtome and which is resistant to histological stains and to degradation from chemicals used to fix and process tissue; and (ii) a plurality of support members arranged on the base member in a predetermined spaced relationship, each of the support members having a stem portion and a head portion, wherein the stem portion has a proximal end attached to the base member and a distal end attached to the head portion, and further wherein the head portion is dimensioned to engage and retain a tissue sample during processing thereof.

A second embodiment of the present invention is directed to a method of preparing a tissue sample for examination, the method comprising: (i) obtaining a sample of tissue; (ii) placing the sample of tissue in a tissue sample support and orientation device; and (iii) processing the sample of tissue as required for examination, wherein the tissue sample support and orientation device comprises: (i) a base member formed of a material which can be sectioned in a microtome and which is resistant to histological stains and to degradation from chemicals used to fix and process tissue; and (ii) a plurality of support members arranged on the base member in a predetermined spaced relationship, each of the support members having a stem portion and a head portion, wherein the stem portion has a proximal end attached to the base member and a distal end attached to the head portion, and further wherein the head portion is dimensioned to engage and retain a tissue sample during processing thereof.

It is to be understood that both the foregoing general description and the following detailed description are exemplary and explanatory only and are intended to provide further explanation of the invention as claimed.

### BRIEF DESCRIPTION OF THE FIGURES

FIG. 1 is a front view of a first embodiment of a tissue sample support and orientation device of the present invention.

FIG. 2 is a side view of the embodiment illustrated in FIG. 1.

FIG. 3 is a top view of the embodiment of the tissue sample support and orientation device illustrated in FIGS. 1 and 2.

### DETAILED DESCRIPTION OF PREFERRED EMBODIMENTS

The present invention will be described in more detail with reference to the embodiments shown in the attached drawings.

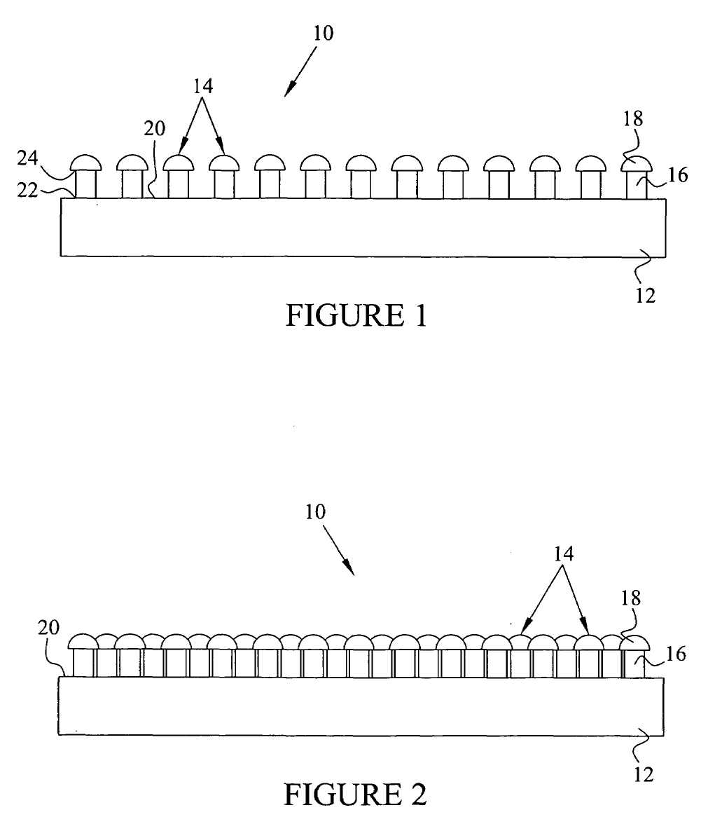

Referring to the drawings, FIGS. 1, 2 and 3 show a tissue sample support and orientation device 10 according to a first embodiment of the present invention. Broadly, this tissue sample support and orientation device comprises: (i) a generally flat base member 12; and (ii) a plurality of support members 14 arranged on the base member 12 in a predetermined spaced relationship.

According to certain preferred embodiments, the support members 14 may be arranged in substantially parallel rows on the base member 12. According to alternative, but equally preferred, embodiments, the support members may be arranged in staggered rows on base member 12. The base member 12 may be of any suitable dimensions for the intended use thereof. For example, when the inventive tissue sample support and orientation device is to be used in conjunction with a standard tissue cassette, the inventive tissue sample support and orientation device is preferably dimensioned to fit within such a standard tissue cassette without any bending, folding or cutting. Illustrative values for the dimensions of base member 12 in such an embodiment are about 25 mm×25 mm×1 mm.

The rows of support members are generally spaced apart a suitable distance for their intended purpose. That is, the rows of support members are spaced apart a sufficient distance to allow a tissue sample to be placed between two adjacent rows and to maintain that tissue sample in the same orientation during further processing, while not adversely affecting the tissue sample. In any of the embodiments of the present invention, the support members within any particular row may be a uniform or a varying distance from one another. Preferably, at least a plurality of support members within a particular row are a uniform distance from one another.

In practice, the rows of support members are preferably spaced between 1.0 and 2.5 mm apart, e.g. 1.2 mm, 1.6 mm, 1.8 mm or 2.0 mm apart. Within a given row, the support members are preferably spaced between 0.1 and 1.0 mm apart, e.g. 0.2 mm, 0.3 mm or 0.5 mm apart. One skilled in the art may determine the particular dimensions of the spaced relationship of the support members empirically, based, for example, on the size, or sizes, of the tissue sample(s) to be examined.

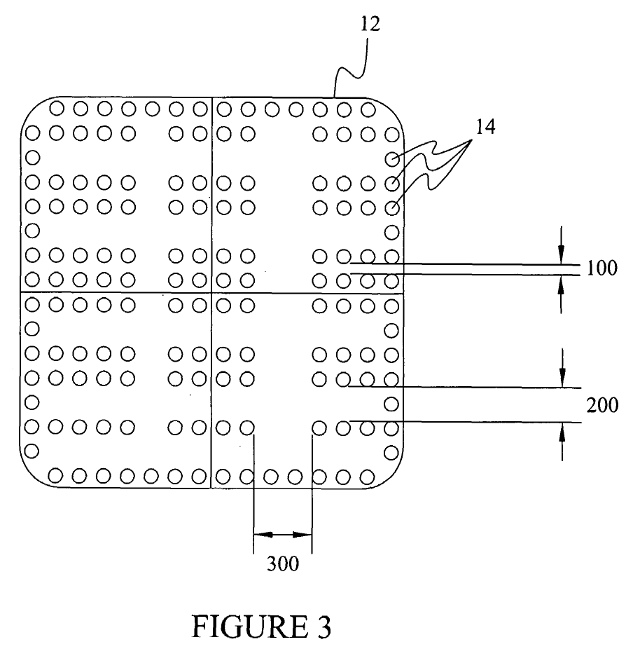

Referring to FIG. 3, a preferred exemplary arrangement of a plurality of support members 14 on a base member 12 is illustrated. According to this embodiment, the support members 14 are arranged in a plurality of substantially parallel rows along the directions of both the x-axis and the y-axis of the base member 12. According to this illustrative embodiment, the base member 12 may be about 25 mm×25 mm and the parallel rows of support members 14 are separated along the x-axis by two possible spacings—one spacing being sufficient to receive a tissue sample from a 14 gauge needle (100) and the other spacing being sufficient to receive a tissue sample from a 16 gauge needle (200). In addition, a third spacing is provided along the y-axis (300), this one being sufficient to receive a tissue sample from an 18 gauge needle. According to certain preferred embodiments of this exemplary arrangement, a single, continuous row of support members is provided around the perimeter of the base of the tissue sample support and orientation device to facilitate retention of the tissue sample(s) during subsequent processing.

Each of the support members 14 has a stem portion 16 projecting substantially upright from the base 12, and a head portion 18 formed at a distal end of the stem portion 16. The head portion 18 is dimensioned to engage and retain a tissue sample during processing. The stem portion 16 has a proximal end 22 connected to the major surface 20 of the base 12, and a distal end 24 connected to the head portion 18.

As noted above, the tissue sample support and orientation device according to the present invention may have any suitable dimensions and/or shape. For example, in the tissue sample support and orientation device 10, the base member 12 may be formed in any suitable dimension and shape that can firmly support the support members 14, such as a rectangular, circular, or elliptical shape. Preferably, base member 12 is approximately square in shape. The thickness of the base member 12 is preferably in a range of 0.5 mm through 5.0 mm, although this may be varied as desired depending upon the particular use of the inventive device. According to particularly preferred embodiments, the inventive tissue sample support and orientation device is dimensioned such that it can fit within a tissue cassette for further processing.

The stem portion 16 of the support member 14 may have various shapes, such as a cylindrical, prism or frustoconical shape, and more than one stem portion may be provided for each head portion 18. Further, a radiused corner having a predetermined radius of curvature may be provided to a junction area between the proximal end 22 of the stem portion 16 and the major surface 20 of the base member 12, for attenuating a stress concentration caused by the deflection of a support member 14. The stem portion 16 is preferably between 0.1 and 1.0 mm in height (i.e. from the base member 12 to the bottom of head portion 18) and the head portion 18 between 0.1 and 0.5 mm high when viewed from the side as in FIGS. 1 and 2.

According to certain preferred embodiments of the present invention, the stem portion 16 is a cylindrical shape having a diameter preferably between 0.1 and 1.0 mm. In such embodiments, the stem portion 16 may have a uniform diameter along its entire length or the diameter may vary. According to certain preferred embodiments, the diameter of the stem portion 16 varies along its length to form one or more grooves to facilitate the positioning of a tissue sample.

The head portion 18 may have various shapes besides the hemispherical shape illustrated in FIGS. 1 and 2. Illustrative examples of suitable shapes include, but are not limited to, shapes such as hook, conical, cylindrical, spherical, pyramidal and hemispherical (umbrella) shapes. Asymmetrical shaped head portions 18 (e.g. cones, pyramids and hemispheres) may be formed in any orientation on the stem portion 16, e.g. if the head portion 18 is a conical shape, the stem portion 16 may be joined to the apex of the cone, the base of the cone or the side of the cone. The size of the head portion 18 must be sufficient to retain a tissue sample when placed in the inventive tissue sample support and orientation device. According to certain preferred embodiments, when viewed from above (as in FIG. 3), the head portion 18 is preferably not more than 0.2 mm in any direction.

According to certain preferred embodiments, for a support member 14 having a cylindrical stem portion 16 and a hemispherical head portion 18, the minimum diameter of the stem portion 16 is preferably in a range of 20% to 60% of the maximum diameter of the head portion 18, in order to obtain sufficient engagement-retaining force on the tissue sample. Also, the peripheral edge of the head portion 18 is preferably formed with no sharp-edges, for reducing possible damage to the tissue sample when the head portion is engaged therewith.

The tissue sample support and orientation device according to the present invention may be made of various materials. Suitable materials include those that are resistant to histological stains and to degradation from chemicals used to fix and process tissue, and that can be sectioned in a microtome. Suitable materials are known and available to those skilled in the art and include, for example, silicone polymers.

For example, the base member 12 of the tissue sample support and orientation device may be formed of a polymeric material, such as silicone, polyamide, polyester, polypropylene, polyethylene, or polyacetal, or a metallic material, such as aluminum. The base member 12 may also be made from a combination of two or more materials. Silicone, polyamide and polyethylene are among the preferred materials because of the excellent thermal durability, mechanical strength and/or adaptability to injection-molding. The base member 12 also preferably includes a plurality of holes and/or slits dimensioned to permit liquid to pass through.

The head portion 18 and the stem portion 16 of the support members may be made of the same material or different materials. The head portion 18 and the stem portion 16 of the support members may also each be made of a single material or a combination of two or more materials. Suitable materials are known and available to those skilled in the art. The material(s) in the support member 14 may be the same material as that of the base member 12, or different material(s). Alternatively, the stem portion 16 of the support member 14 may be made of a high-strength material different from the material of the base member 12 and the head portion 18, e.g. to improve durability. According to a particularly preferred embodiment, the base member 12 and the support members 14 are made from the same material.

The base member 12 and the support members 14 are preferably each made of a material, or combination of materials, that are sufficiently flexible to permit the inventive tissue sample support and orientation device to be bent (or otherwise manipulated) so as to facilitate placement of the tissue sample in the device, and yet then return to substantially the same shape as it was before such bending or manipulation. For example, the base member 12 and the support members 14 may have a bending modulus of elasticity in a range of 1,000 kgf/cm² through 100,000 kgf/cm² according to a measurement based on ASTM testing method D790. Also, it is possible to add a plasticizer or rubber, or to add a reinforcing member, such as carbon fibers or glass fibers, to the polymeric material, so as to suitably modify or change the elastic modulus of the base member 12 and the support members 14 as needed.

According to certain preferred embodiments, the material(s) used for the base member and/or the support members has a net specific density that promotes the separation of the material(s) from the tissue sample when the tissue sample and inventive tissue sample support and orientation device are subjected to further processing. For example, according to certain preferred embodiments, further processing of the sample may involve the tissue sample and inventive tissue sample support and orientation device being floated on and/or placed in a liquid bath. This liquid bath would be designed to reduce and/or eliminate the bonds and/or other interactions that hold the material(s) and the tissue sample(s) together, e.g. the liquid would be composed of one or more chemicals capable of reducing and/or eliminating these bonds and/or other interactions or the liquid would be maintained at a temperature sufficient to reduce and/or eliminate these bonds and/or other interactions. As a result, the material(s) can sink into the liquid or otherwise separate from the tissue sample. This can be a particular advantage when the material(s) are potentially visually distracting to those subsequently examining the tissue sample.

The tissue sample support and orientation device according to the present invention may be formed by various methods known and available to those skilled in the art. To easily form support members 14 having unique shapes, it is advantageous to integrally mold the base and the headed elements by an injection molding process using a destructible stem mold, as is disclosed, for example, in U.S. Pat. No. 5,242,646, the contents of which is incorporated herein by reference.

The tissue sample support and orientation device of the present invention is particularly useful for processing biopsy tissue samples for analysis.

Broadly, the method of preparing biopsy tissue samples for histological examination comprises: (a) removing a tissue sample from a patient; (b) placing the tissue sample onto a support and orientation device of the present invention such that the support members engage and retain the sample; (c) processing the tissue sample. Such processing may include subjecting both the inventive support and orientation device and the tissue sample immobilized thereon to a process for replacing tissue fluid with wax and thereby impregnating the tissue sample with wax, and then embedding the tissue sample and the inventive support and orientation device in a wax mold to form a solid block of wax. Subsequently, a microtome may then be used to slice the solid block of wax (including the tissue sample and inventive support and orientation device) into thin sections which can be used for further examination and analysis.

The foregoing description and the following examples are illustrative only and are not intended to limit the scope of the invention as defined by the appended claims. It will be apparent to those skilled in the art that various modifications and variations can be made in the methods of the present invention without departing from the spirit and scope of the invention. Thus, it is intended that the present invention cover the modifications and variations of this invention provided they come within the scope of the appended claims and their equivalents.

What is claimed is:

1. A tissue sample support and orientation device comprising:
(i) a base member, said base member being formed of one or more materials which can be sectioned in a microtome and which is resistant to histological stains and to degradation from chemicals used to fix and process tissue; and
(ii) a plurality of support members arranged on said base member in a predetermined spaced relationship, each of said support members having a stem portion and a head portion and being formed of one or more materials which can be sectioned in a microtome and which is resistant to histological stains and to degradation from chemicals used to fix and process tissue,
wherein said stem portion has a proximal end attached to said base member and a distal end attached to said head portion, and
further wherein said head portion is dimensioned to engage and retain a tissue sample during processing thereof.

2. The tissue sample support and orientation device according to claim 1, wherein said one or more materials includes at least one polymer selected from the group consisting of silicone, polyamide, polyester, polypropylene, polyethylene and polyacetal.

3. The tissue sample support and orientation device according to claim 2, wherein said base member comprises silicone.

4. The tissue sample support and orientation device according to claim 1, wherein said support members comprise silicone.

5. The tissue sample support and orientation device according to claim 1, wherein said one or more materials are sufficiently flexible to permit said inventive tissue sample support and orientation device to be bent (or otherwise manipulated) and yet then return to substantially the same shape.

6. The tissue sample support and orientation device according to claim 1, wherein each head portion of said plurality of support members has substantially the same shape.

7. The tissue sample support and orientation device according to claim 5, wherein said shape is selected from the group consisting of a hook shape, a conical shape, a cylindrical shape, a spherical shape, a pyramidal shape and a hemispherical (umbrella) shape.

8. The tissue sample support and orientation device according to claim 1, wherein said predetermined spaced relationship is a plurality of substantially parallel rows.

9. The tissue sample support and orientation device according to claim 8, wherein the spacing between any three of said substantially parallel rows is substantially uniform.

10. The tissue sample support and orientation device according to claim 8, wherein the spacing between any three of said substantially parallel rows is not substantially uniform.

11. The tissue sample support and orientation device according to claim 1, wherein said base member includes a plurality of holes and/or slits.

12. The tissue sample support and orientation device according to claim 1, wherein said stem portion has a substantially cylindrical shape.

13. The tissue sample support and orientation device according to claim 12, wherein said stem portion has a substantially uniform diameter along its entire length.

14. The tissue sample support and orientation device according to claim 12, wherein said stem portion has a varying diameter along its entire length.

15. The tissue sample support and orientation device according to claim 14, wherein said stem portion has one or more grooves.

16. The tissue sample support and orientation device according to claim 1, wherein said stem portion has a substantially cylindrical shape and said head portion has a conical shape.

17. The tissue sample support and orientation device according to claim 12, wherein the minimum diameter of said stem portion is between 20% and 60% of the maximum diameter of said head portion.

18. The tissue sample support and orientation device according to claim 1, wherein said one or more materials have a net specific density that facilitates separation of said one or more materials from said tissue sample during subsequent processing thereof.

19. A method of preparing a tissue sample for examination, said method comprising:
(a) obtaining a tissue sample from a patient;
(b) placing said tissue sample in a tissue sample support and orientation device according to claim 1; and
(c) processing said tissue sample for further examination.

20. The method according to claim 19, wherein said processing step (c) comprises:
(c1) subjecting both said tissue sample support and orientation device and said tissue sample immobilized thereon to a process for replacing tissue fluid with wax and thereby impregnating said tissue sample with wax; and
(c2) embedding said tissue sample and said tissue sample support and orientation device in a wax mold to form a solid block of wax; and
(c3) using a microtome to slice said solid block of wax into thin sections, each one containing a portion of said tissue sample which can be used for further examination and analysis.
